(19) United States
(12) Patent Application Publication
(10) Pub. No.: US 2013/0018326 A1
(43) Pub. Date: Jan. 17, 2013

(54) MEDICAL FLUID INJECTION DEVICE AND SYSTEM

(71) Applicant: Michael D. Hooven, Cincinnati, OH (US)

(72) Inventor: Michael D. Hooven, Cincinnati, OH (US)

(73) Assignee: Enable Injections, LLC, Cincinnati, OH (US)

(21) Appl. No.: 13/637,756

(22) PCT Filed: Mar. 31, 2011
(86) PCT No.: PCT/US11/30748

§ 371 Date: Sep. 27, 2012

Related U.S. Application Data
(60) Provisional application No. 61/320,170, filed on Apr. 1, 2010.
(60) Provisional application No. 61/326,492, filed on Apr. 21, 2010.

(51) Int. Cl. A61M 5/32; A61J 1/20

(52) U.S. Cl. 604/198; 604/415

(57) ABSTRACT

A medical fluid injection device and/or medical fluid vial are disclosed. The medical fluid vial includes an outer body with a mouth and an inner member having a resilient expandable container portion and an access portion normally sealing the container portion. The injection device has a housing or needle holder mounting an access member for accessing the vial contents and a needle for injection into a patient. The housing may include a needle shield movable relative to the needle holder.

This application claims the benefit of U.S. Provisional Applications Nos. 61/326,492, filed Apr. 21, 2010, and 61/320,170 filed Apr. 1, 2010, both of which are hereby incorporated by reference.

The present subject matter relates to medical fluid injection devices, systems and methods for transdermal injection of vaccines, drugs and other medical fluids into a patient.

It is common to pre-package vaccines, drugs and other medicinal fluids (referred to generally as “medical fluids”) in glass vials sealed by a butyl or silicone septum. To inject the vial contents into a patient, the medical personnel typically must retrieve a separate syringe, insert the needle of the syringe into the vial, withdraw the desired dosage of medical fluid, inject the patient and dispose of the syringe.

Efforts have been made to provide integrated drug and injection devices in the form of pre-filled syringes, implantable drug pumps and disposable unit dose devices such as found in published applications WO2005/079440; WO2005/079441; WO2004/024211; and WO2008/036509 and in U.S. Pat. Nos. 4,031,889, 5,616,132 and 6,045,534, all of the above being hereby incorporated by reference herein. There remains a need, however, for alternative, improved, simplified and/or lower cost injection devices, methods and/or systems.

### SUMMARY

In accordance with one aspect of the present disclosure, a single use medical fluid reservoir or vial is disclosed having an outer body with a mouth, usually at one end thereof, and an inner reservoir or vial member comprising a resilient expandable container portion located within the vial body for containing medical fluid and exerting pressure thereon, and an access portion disposed within the vial mouth. The access portion normally seals the container portion and provides an access area for withdrawing medical fluid of the container portion.

In another aspect, the reservoir or vial above may be used with a needle holder that mounts a vial access member and an injection needle, and is configured to receive the vial for movement between an injection position and an injection position in which the access member penetrates the access portion to access the medical fluid in the container portion and the needle extends for insertion into a patient.

The access portion may be in the form of a standard septum and may include a pre-formed, normally closed aperture or slit for receiving a blunt cannula to penetrate the access portion or septum. Alternatively, the access potion may be solid, such as a fully closed septum, that can be penetrated by a needle or other penetrating device. In either version, the medical fluid access portion may also include a filling orifice for introducing medical fluid into the container portion and a plug, which may also be pierceable, disposed within the filling orifice and sealing the orifice after filling.

In accordance with another aspect of the present disclosure a medical fluid injection device or system is provided for injecting medical fluid under pressure from a vial, through a needle and into a patient. The vial may be such as described above, but the injection device is not limited to such a vial. The injection device includes a vial-receiving housing configured to allow a vial to be accessed or advanced through a plurality of stages or positions such as, for example, a loading or loaded stage, an intermediate or pre-injection needle-extended stage or position, and an injection stage or position, and then a retracted stage or position.

In the loaded stage, the injection needle is shielded by the housing from inadvertent user contact and the contents of the vial remain sealed and unaccessed. In the loaded stage the housing is preferably but not necessarily configured to substantially prevent withdrawal of a vial therefrom, and alternatively or additionally may be configured to prevent inadvertent advancement from the loaded stage toward the intermediate stage. The housing may include a separate user releasable lock member which is broadly defined to include any preventing or securing member or means and includes an interfering member or surface that is integrated with the device or its packaging or separable therefrom, to prevent inadvertent advancement from the loaded position toward the needle-extended position. As noted above, such a lock may be in the form of a user-removable pin or ring that physically prevents such advancement. Alternatively, the device may be supplied in a package that employs an interfering portion that prevents such inadvertent advancement.

In any event, upon user election or release the device and/or vial may be advanced to an intermediate or needle-extended stage. In this stage, the needle extends from the housing for insertion into a patient, but preferably the vial remains sealed and the contents unaccessed by the access member. The housing may also be configured to prevent withdrawal or retraction of a vial from the intermediate stage position.

When the vial is advanced from the intermediate stage to the injection stage, the vial access portion or septum is penetrated by an access member, such as a needle or blunt cannula, allowing medical fluid, under pressure from within the vial, to flow through the injection needle and into the patient. Preferably, the vial and needle are releasably held by the housing at the injection stage and, upon user release, are automatically returned by a biasing force to a retracted stage or position where the needle is again shielded by the housing. Although, described with four stages, more or fewer stages or positions may be employed. For example, only two advancing stages may be used and the intermediate stage eliminated, or the intermediate and injection stage be combined into a single stage or position and/or the fourth stage or position eliminated.

In accordance with another aspect of this disclosure, a medical fluid injection device is disclosed which includes a pre-filled reservoir or vial containing a pressurized medical fluid, such as that described above, and including a normally sealed access port. The medical fluid injection device may include an elongated needle shield that is cooperatively associated with the access port and an administration needle having a proximal end, a distal piercing end, and if desired, an intermediate access port between the proximal end and distal piercing end, with an internal lumen within the needle extending at least between the intermediate access opening and the distal piercing end. The needle is carried within the needle shield and movable between a retracted position fully located within the shield and an injection position in which the intermediate access opening of the needle is in fluid communication with the reservoir to access the contents of the reservoir, and the distal piercing end of the needle extends freely for piercing the skin of a patient. The medical fluid within the reservoir is pressurized so as to flow from the reservoir through the intermediate opening or port and the lumen of the needle when the needle is in the injection position. After injection of the medical fluid, the needle is retractable to a position sufficiently within the shield to avoid inadvertent user contact with the piercing end of the needle. The needle shield may be fixably attached to the reservoir access port or may be separately formed and removably attached to the reservoir access port when needed.

Also, in any of the aspects described above, the needle lumen may be sized, length and diameter, for injecting a dose of medical fluid in less than one minute or for longer time periods to reduce or eliminate pain. Alternatively, or additionally, the pressurization of the medical fluid may be higher for faster injection flow rates or lower for slower injection flow rates. In more specific embodiment, the needle has an outside diameter of at least about 0.2 mm.

The above are just a few of the aspects of the present subject matter, which is described more fully below. It is understood that aspects of the device and system may be used together or separately as the particular design and use determines.

Turning now to the accompanying figures:

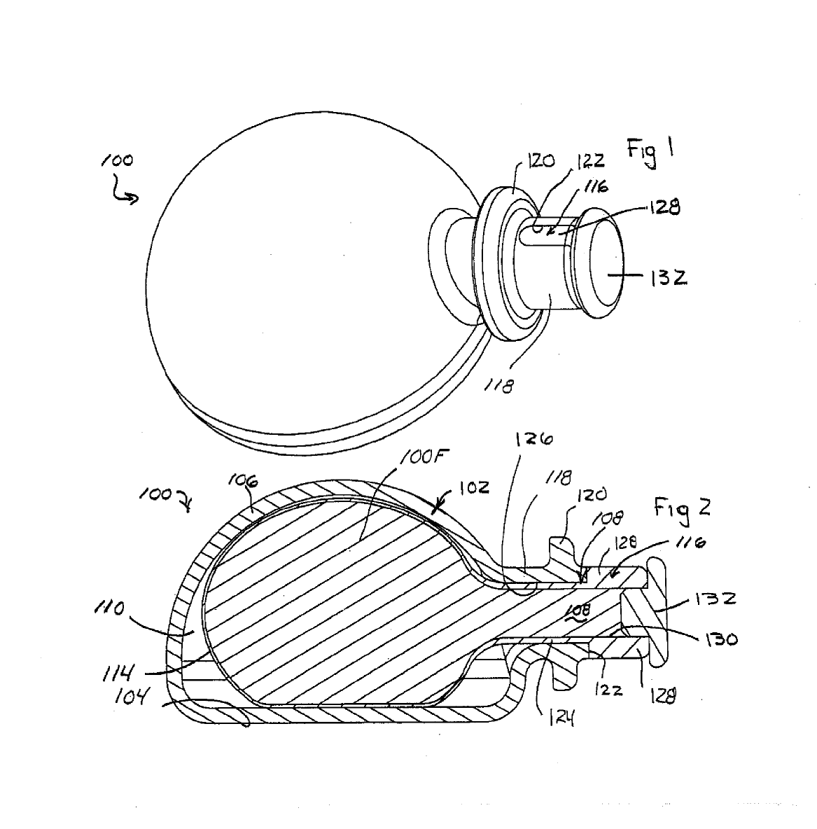

FIG. 1 is a perspective view of a prefilled fluid reservoir or vial substantially filled of a unit-dose of medical fluid under pressure from within the reservoir or vial.

FIG. 2 is a vertical cross-sectional view of the medical fluid reservoir or vial of FIG. 1.

FIG. 3 is a perspective view of one embodiment of a medical fluid injection device, also referred to as a needle assembly, that may be used in association with the vial illustrated in FIGS. 1 and 2.

FIG. 4 is a longitudinal cross-sectional view of the injection device or needle assembly of FIG. 3.

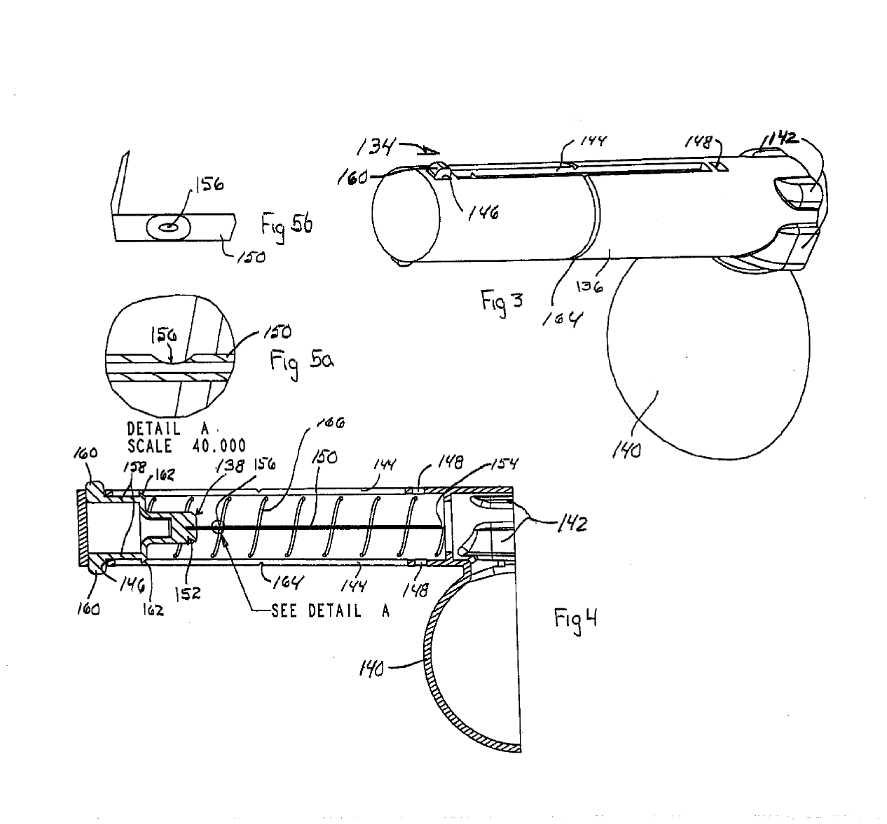

FIG. 5a is a longitudinal cross-sectional view of DETAIL A of the needle in FIG. 4, showing a side intermediate access opening or port in the needle.

FIG. 5b is a partial elevational view of the access opening depicted in DETAIL A of FIGS. 4 and 5a.

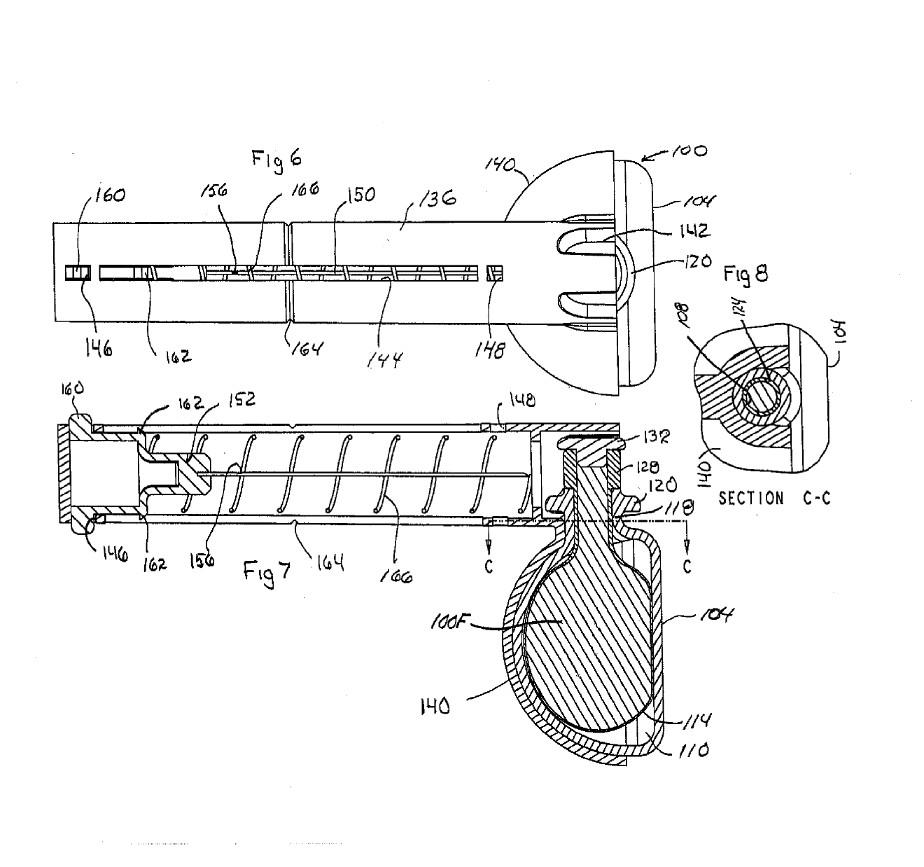

FIG. 6 is an elevational view of the injection device, or needle assembly, of FIGS. 3 and 4 assembled to or in cooperative association with the filled medical fluid reservoir or vial of FIGS. 1 and 2.

FIG. 7 is a longitudinal cross-sectional view of the assembled needle assembly and vial of FIG. 6.

FIG. 8 is a partial cross-sectional view taken along line c-c of FIG. 7.

FIG. 9 is a perspective view of the needle assembly and vial of FIGS. 6 and 7, with the needle in an advanced or injection position for injection of medical fluid into a patient.

FIG. 10 is a top view of the assembly of FIG. 9.

FIG. 11 is an end view of the needle assembly and vial of FIG. 9.

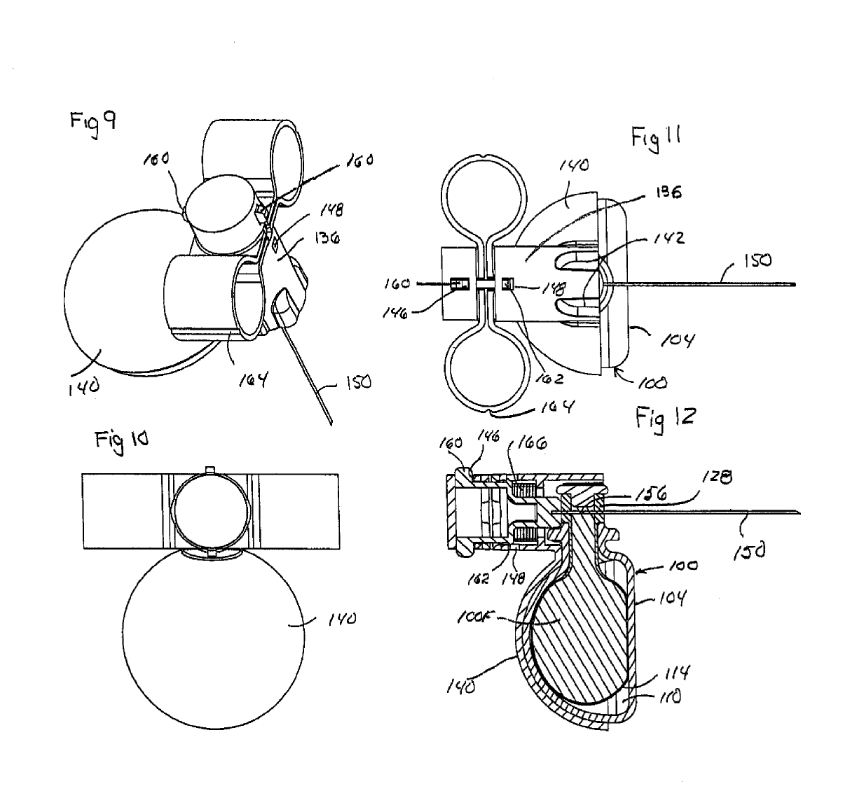

FIG. 12 is a vertical cross-sectional view of the combined needle assembly and vial of FIG. 11.

FIGS. 13 is a perspective view showing the combined needle assembly and vial of FIGS. 6 and 7 in a retracted position after the medical fluid has been dispensed into the patient, with the needle in the retracted position within a needle shield.

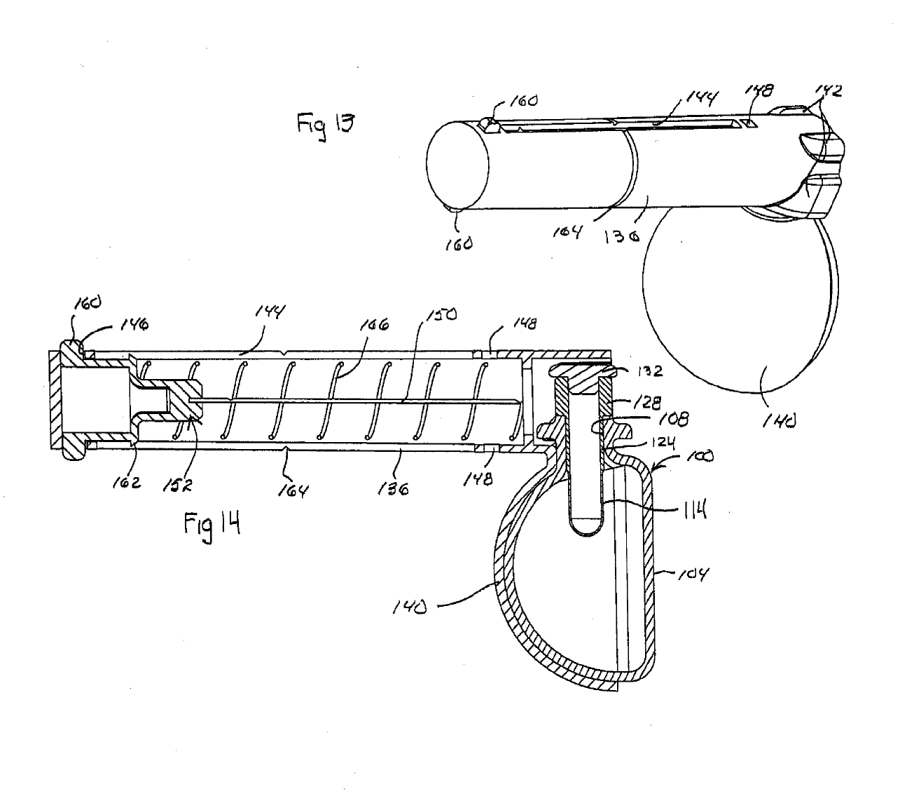

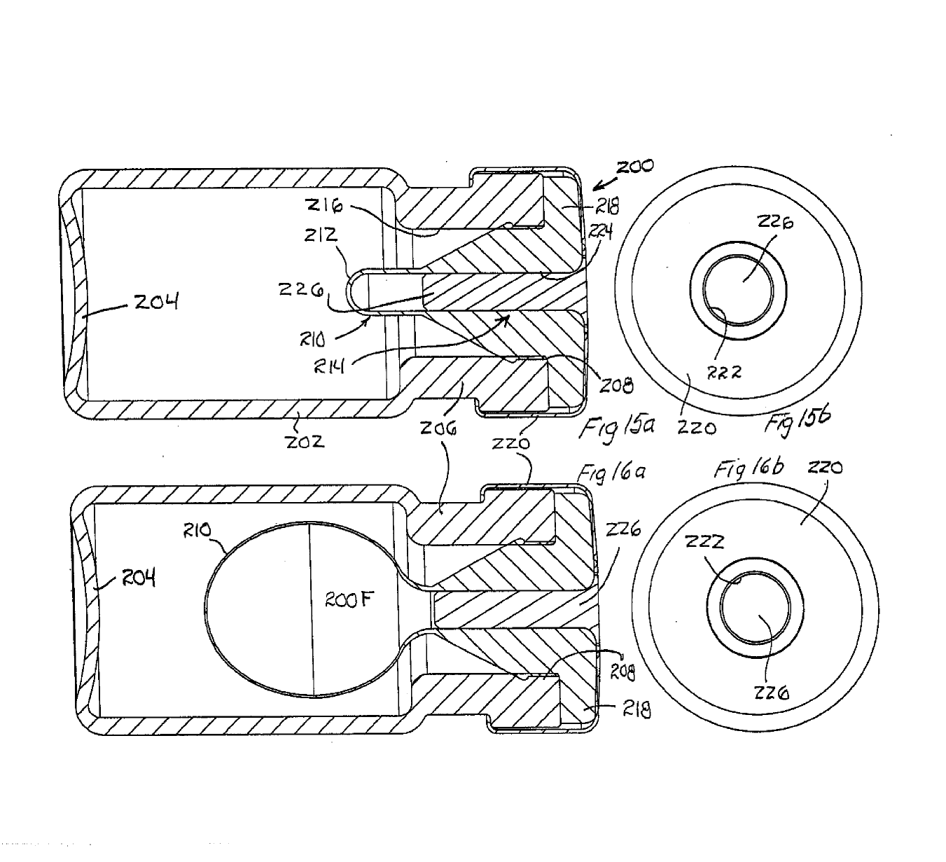

FIG. 14 is a longitudinal cross-sectional view of the combined needle assembly and vial of FIG. 13.

FIGS. 15a and 15b are cross-sectional and end views, respectively, of an alternate configuration of a unit dose medical fluid vial before filling or after withdrawal of medical fluid.

FIGS. 16a and 16b are cross-sectional and end views, respectively, of the vial shown in FIGS. 15a and 15b, but filled with a unit dose of medical fluid.

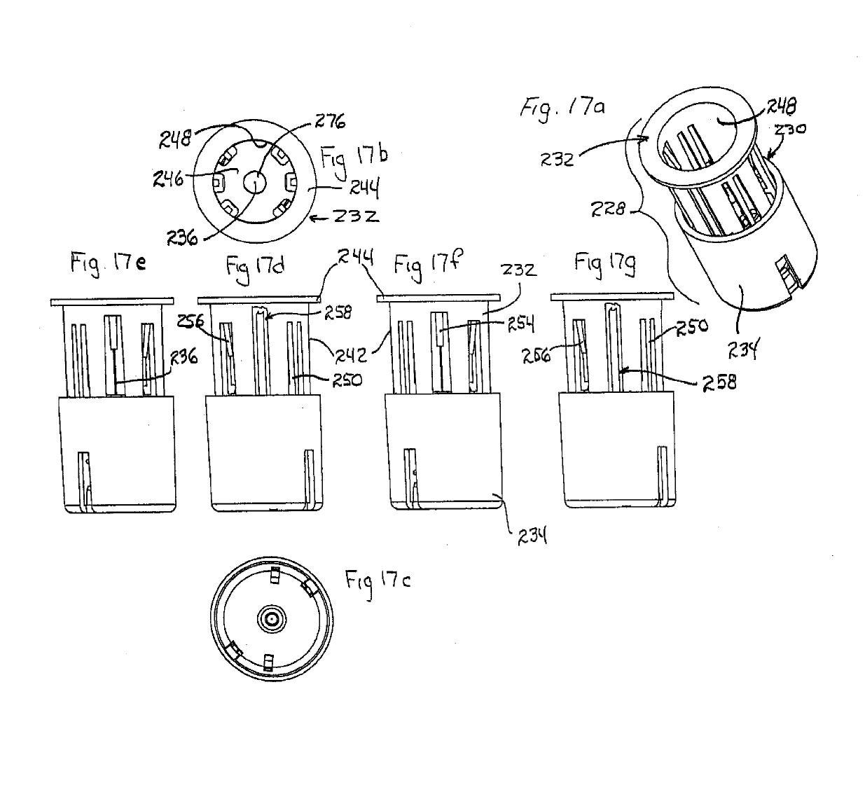

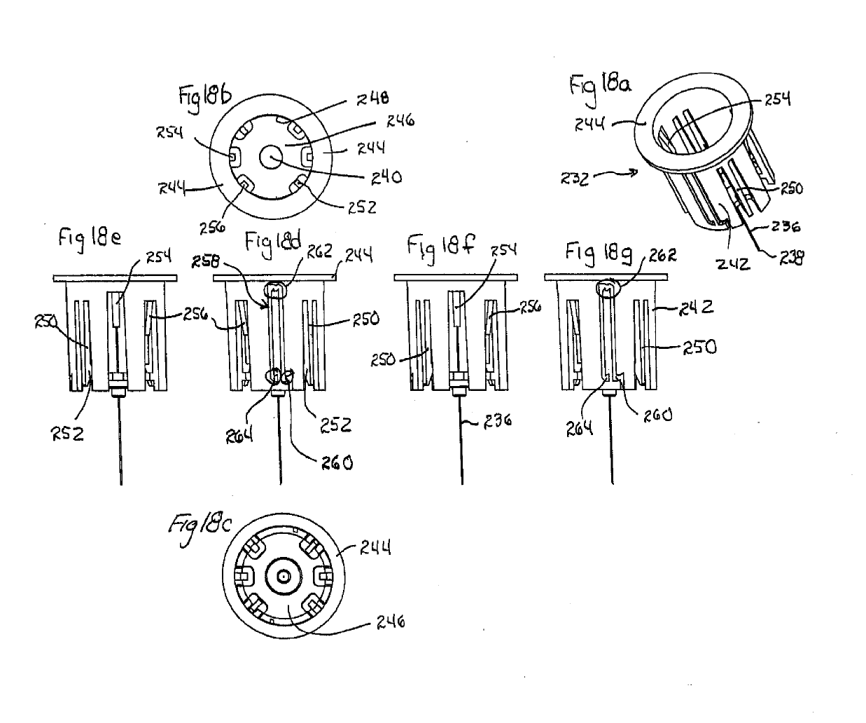

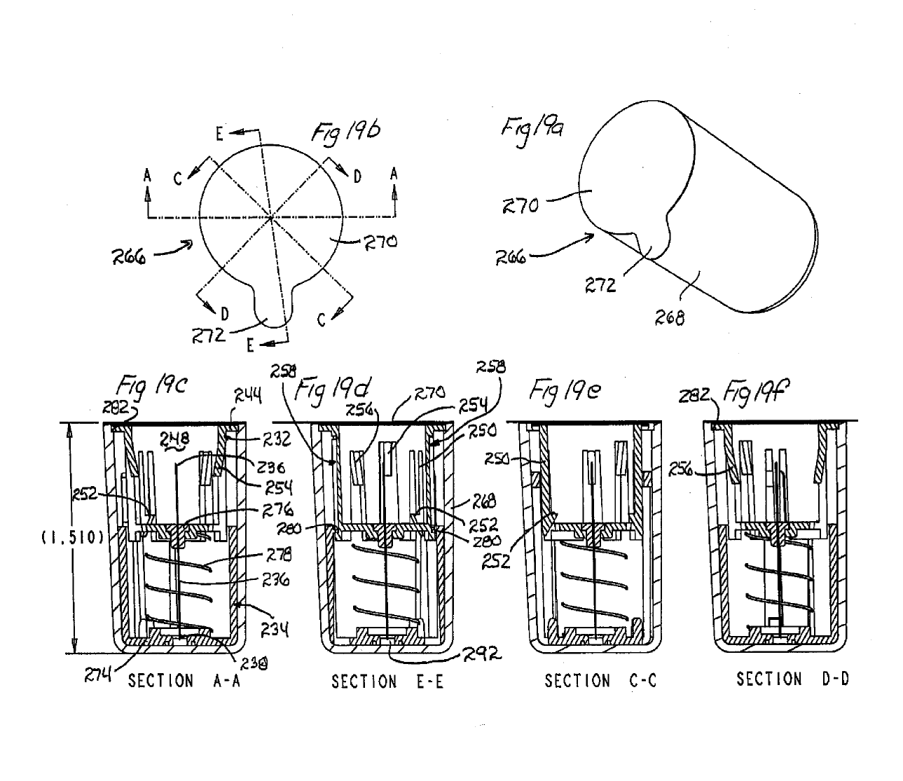

FIG. 17a is a perspective view of a medical fluid injection device or system, also referred to interchangeably as a needle assembly, assembled and before packaging.

FIG. 17b is a top view of the injection device of FIG. 17a, viewing into a vial receiving recess.

FIG. 17c is a bottom view of the injection device of FIG. 17a.

FIGS. 17d-g are front, side and rear elevational views of the injection device of FIG. 17a.

FIGS. 18a is a perspective view of a needle holder component of the device of FIG. 17a.

FIG. 18b is a top view of a needle holder component of FIG. 18a, viewing into a vial receiving recess.

FIG. 18c is a bottom view of the needle holder component of FIG. 18a.

FIG. 18d-g are front, side and rear elevational views of the needle holder component of FIG. 18a.

FIG. 19a is a perspective view of a package unit containing the medical fluid device or system of FIG. 17a within an outer package.

FIG. 19b is an end view of the package medical device unit of FIGS. 19a.

FIG. 19c is a vertical cross-sectional view of the packaged medical device unit, taken along lines A-A of FIG. 19b.

FIG. 19d is a vertical cross-sectional view of the packaged medical device unit of FIG. 17a, taken along line E-E of FIG. 19b.

FIG. 19e is a vertical cross-sectional view of the packaged medical device unit of FIG. 17a, taken along line C-C of FIG. 19b.

FIG. 19f is a vertical cross-sectional view of the packaged medical device unit of FIG. 17a, taken along line D-D of FIG. 19b.

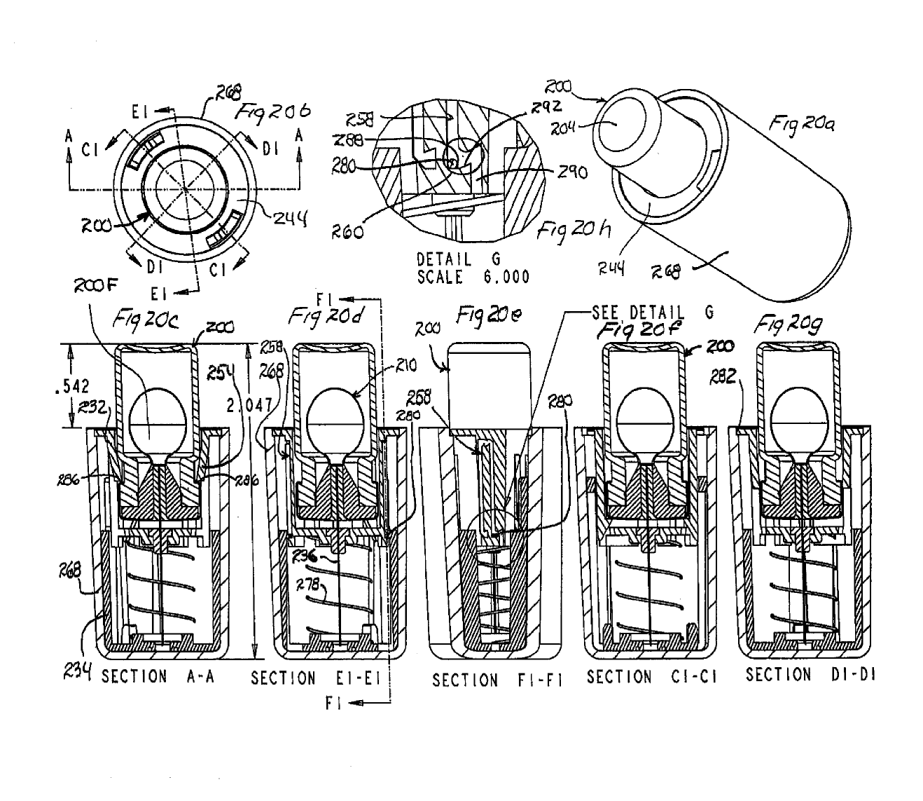

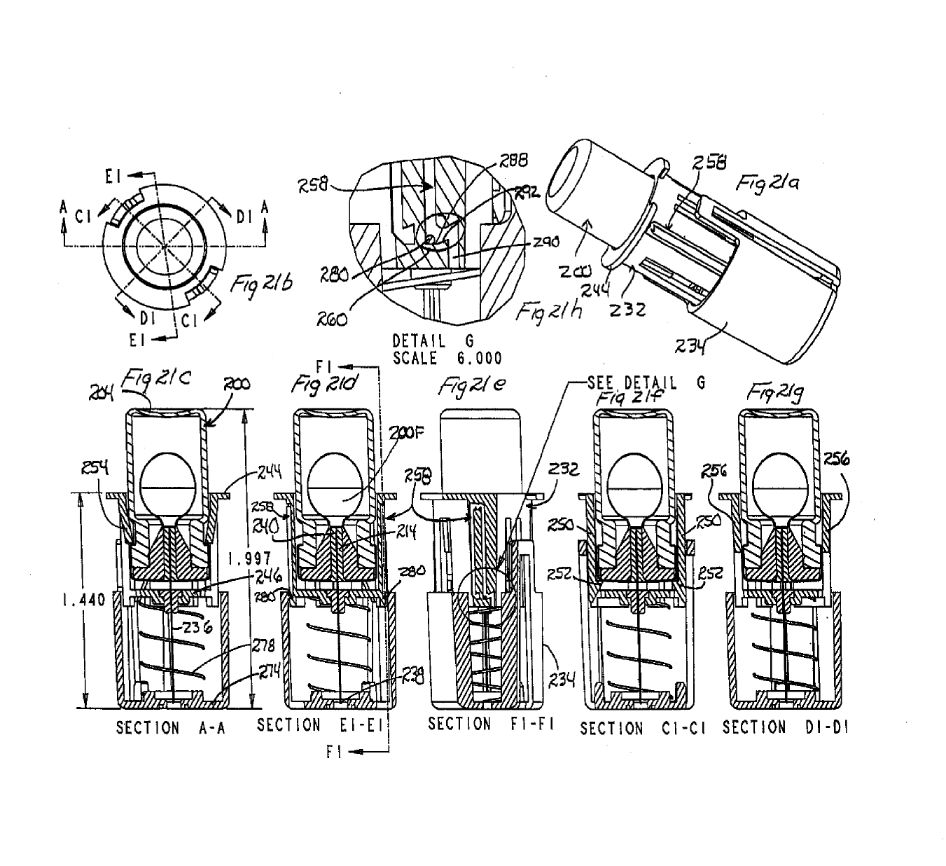

FIGS. 20a-20g illustrate the packaged medical fluid injection system and device of FIGS. 19a-f within the outer package and with an end seal of the package removed and a pre-filled vial of medical fluid inserted to an initial loaded stage or position in a vial receiving recess in the needle holder.

FIG. 20a is a perspective view of a medical fluid injection device or assembly situated within the outer package and having a pre-filled unit dose medical fluid vial inserted to a loaded stage or position in a vial receiving recess therein.

FIG. 20b is an end view of the assembly of FIG. 20a.

FIG. 20c is a cross-sectional view of the assembly of FIGS. 20a and 20b, taken along line A-A of FIG. 20b.

FIG. 20d is a vertical cross-sectional view of the assembly of FIGS. 20a and 20b, taken along the line E-E of FIG. 20b.

FIG. 20e is a vertical cross-sectional view of the assembly of FIGS. 20a and 20b, taken along line F-F of FIG. 20d, to better depict a cam/follower arrangement associated with the device and to show relative movement between a cam track in the needle holder and a cam follower in the needle shield, as described more fully below.

FIG. 20f is a vertical cross-sectional view of the assembly of FIGS. 20a and 20b, taken along line C-C of FIG. 20b.

FIG. 20g is a vertical cross-sectional view of the assembly of FIGS. 20a and 20b, taken along line D-D of FIG. 20b.

FIG. 20h is an enlarged isolated view of the cam track or slot and follower detail of FIG. 20e.

FIGS. 21a-21h are the same views as in FIGS. 20a-20h, but with the medical fluid device or system of FIGS. 20a-20h removed from the container, and with a vial still in the loaded position in the needle holder.

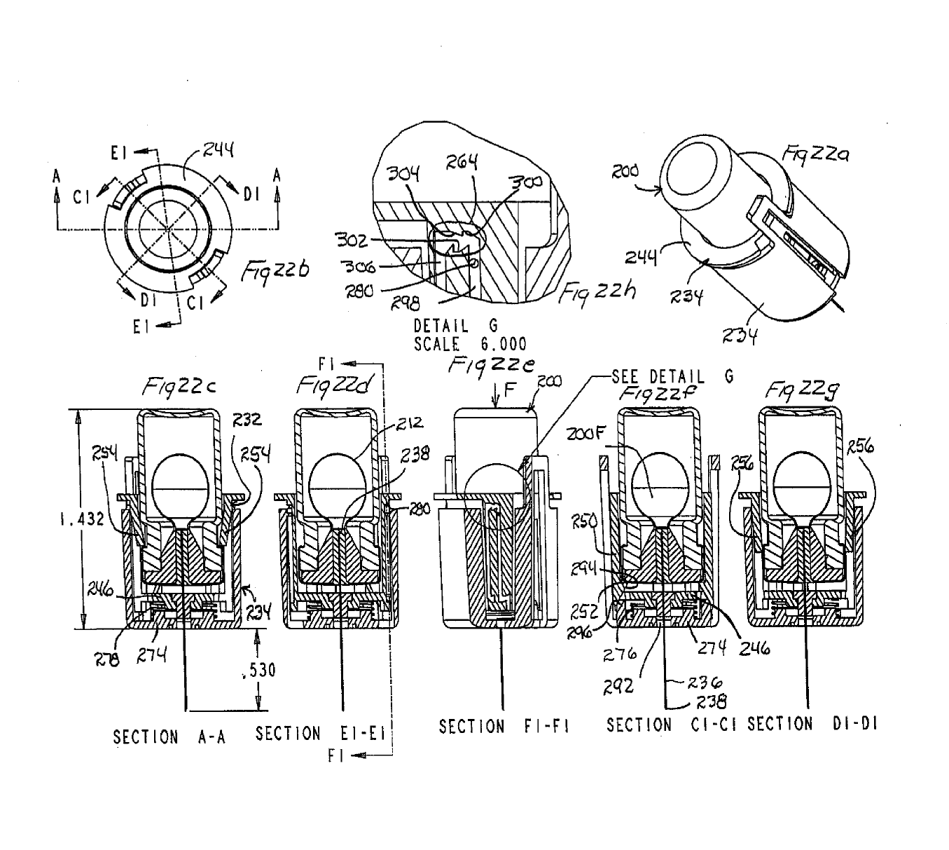

FIGS. 22a-22g are the same views as in FIGS. 21a-21h, but with a vial advanced from the loaded position to an intermediate or pre-injection position or stage in which the vial remains sealed and the needle is extended for injection into a patient.

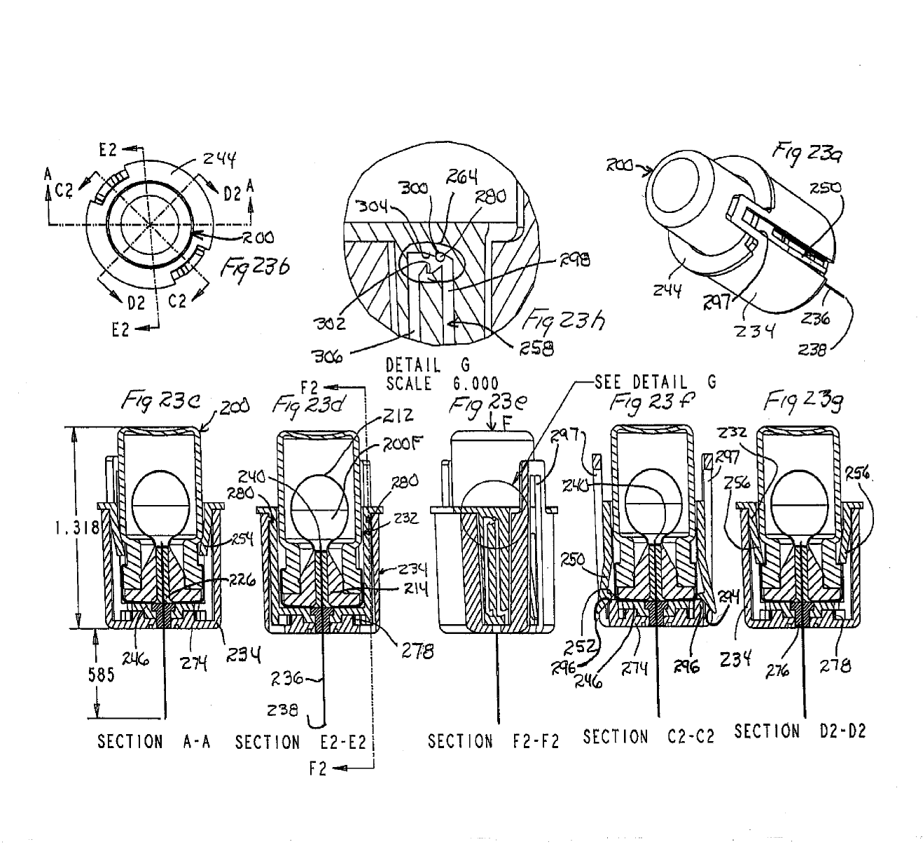

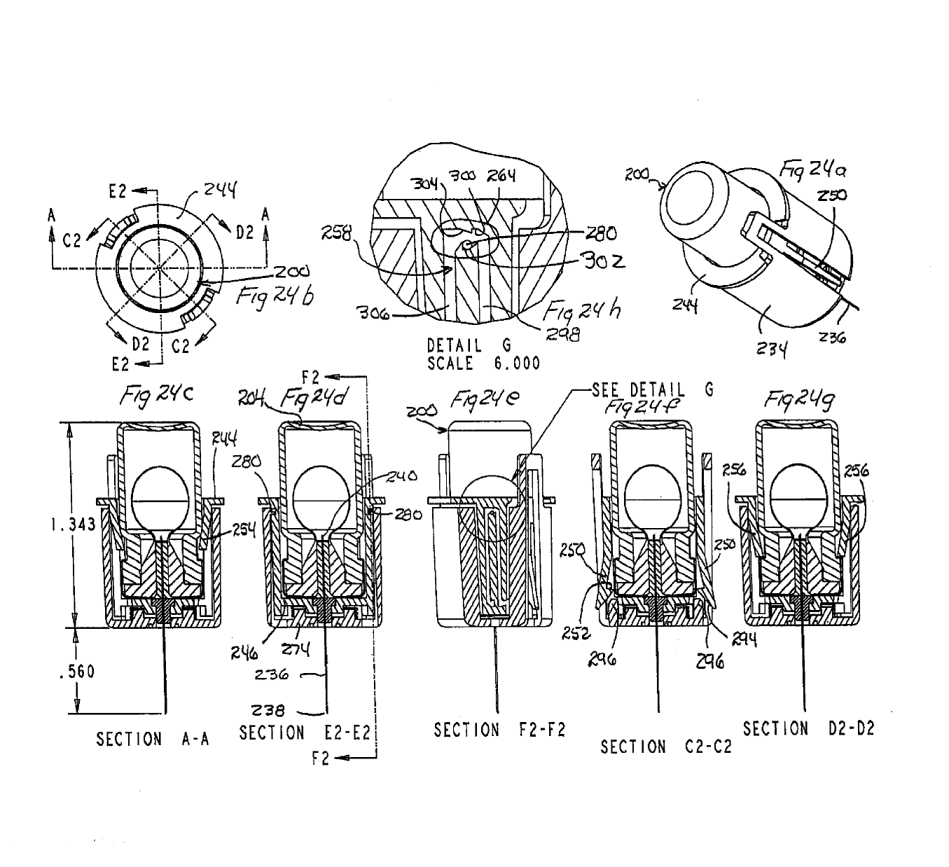

FIGS. 23a-23h are the same view as in FIGS. 22a-22g, but illustrate the injection device with a vial advanced to injection position or stage, with a user actuation force F being applied to the end of the wall, with the vial contents being accessed and medical fluid being injected from the vial into a patient.

FIGS. 24a-24h are the same view as in FIGS. 23a-23h with the assembly in the injection position in which medical fluid in being injected, but after the user advancing force F against the bottom of the vial has been released.

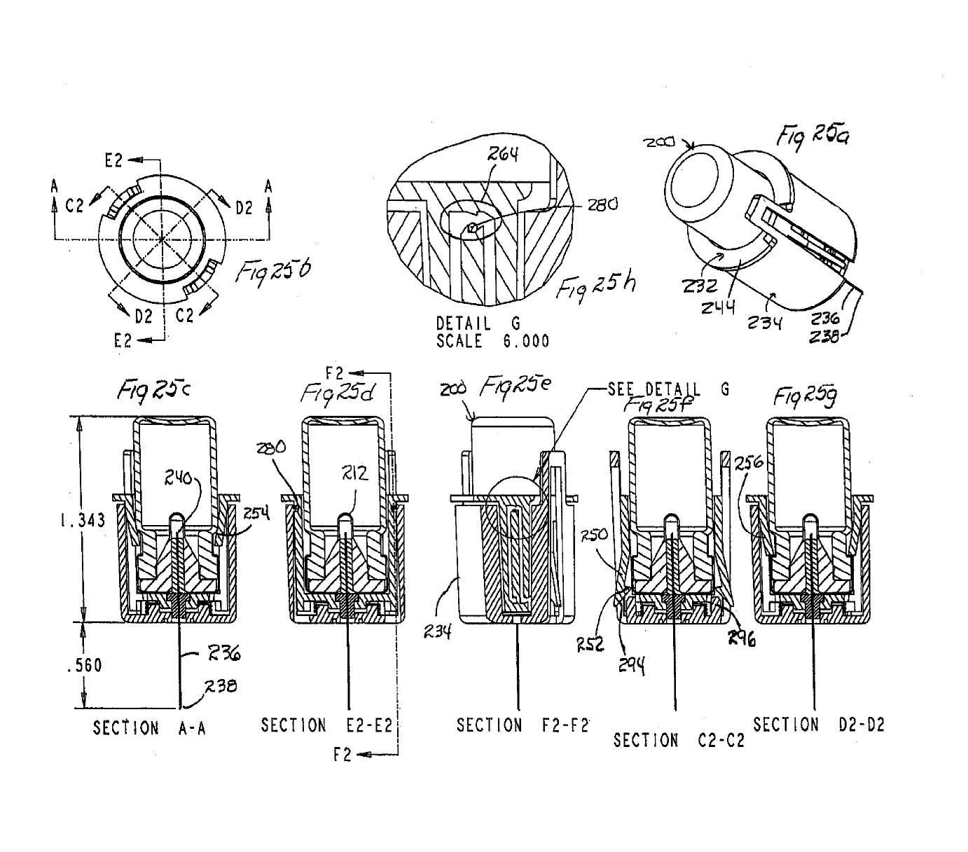

FIGS. 25a-25h are the same view as in FIGS. 24a-24h, with the assembly in the injection position, but after injection of the medical fluid into a patient is complete.

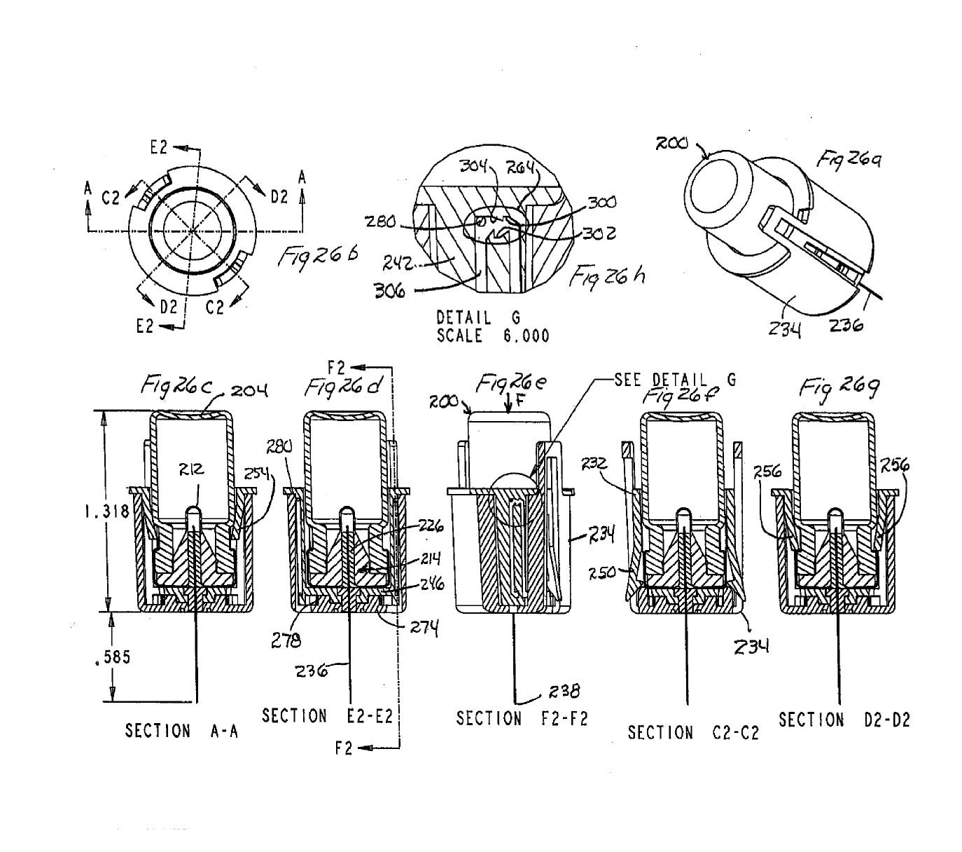

FIGS. 26a-26h are the same view as in FIGS. 25a-25h, with the assembly In the injection position and after injection is completed, but after user force has again been applied to the bottom of the vial.

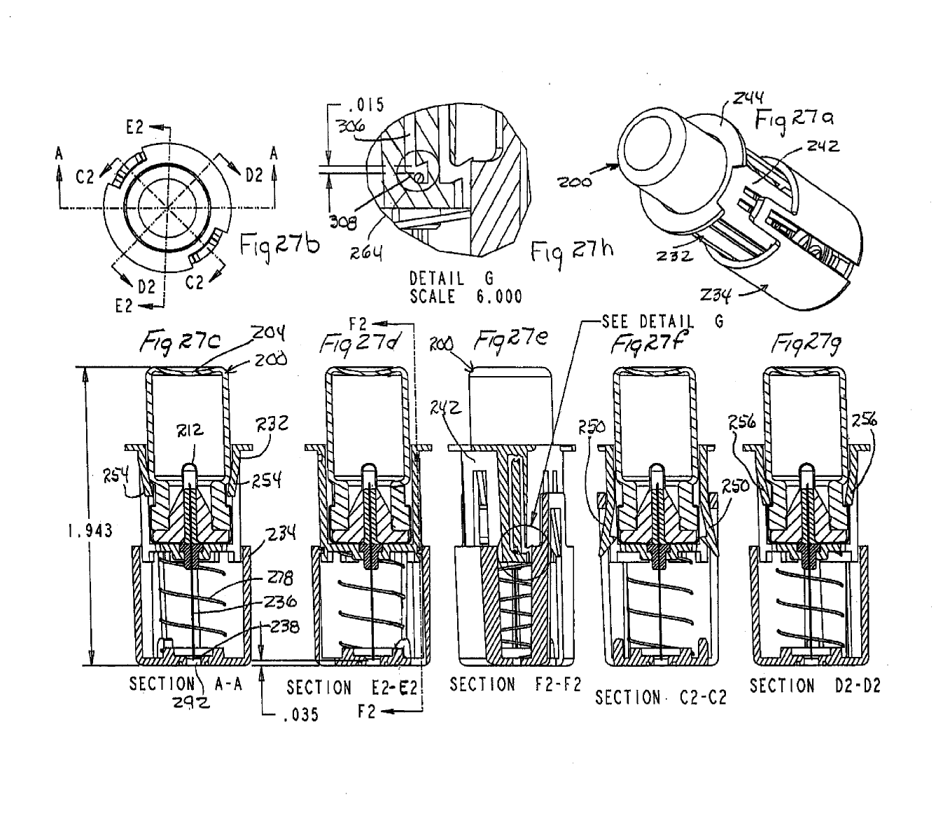

FIGS. 27a-27h are the same view as in FIGS. 26a-26g, except that FIGS. 26a-26h show the assembly after it has moved to a retracted position (in which the needle is located within and protected from inadvertent contact by a needle shield) upon release of user force on the bottom of the vial from the position in FIGS. 26a-g.

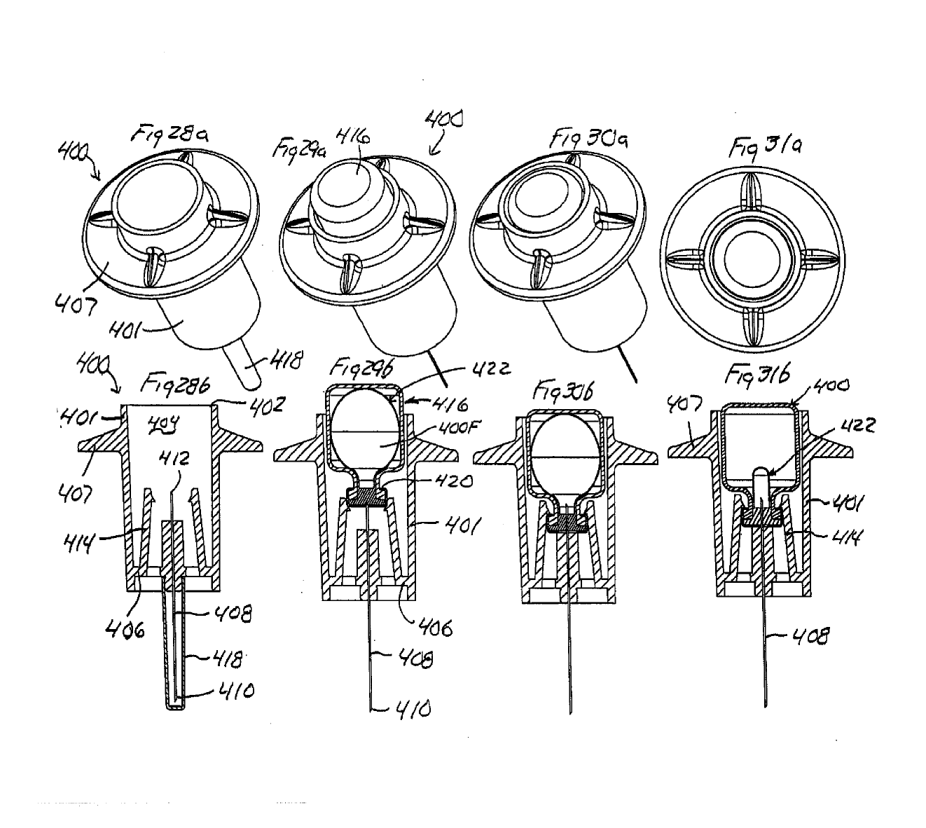

FIG. 28a is a perspective view of a needle holder in accordance with another embodiment of the present subject matter.

FIG. 28b is a vertical cross-sectional view of the needle holder of FIG. 28a.

FIG. 29a is a perspective view of the needle holder of FIGS. 28a-b with a vial similar to FIGS. 15 and 16 inserted to an insertion or loaded position in a vial receiving recess in the holder.

FIG. 29g is a vertical cross-sectional view of the needle holder and vial assembly of FIG. 29a.

FIG. 30a is a perspective view of the needle holder and vial assembly of FIGS. 29a-b with the vial advanced to an access position for injection and before the medical fluid is injected.

FIG. 30b is a vertical cross-sectional view of the needle holder and vial assembly of FIG. 30a.

FIG. 31a is a perspective view identical to FIG. 30a, but after medical fluid has been injected.

FIG. 31b is a vertical cross-sectional view of FIG. 31a.

Turning now to a more detailed description, FIG. 1 is a perspective view of a fluid reservoir or vial 100 suitable for containing a medical fluid such as a vaccine, drug or other medication, diagnostic or therapeutic liquid.

The illustrated fluid reservoir 100 in this embodiment preferably includes a substantially rigid outer housing or body generally at 102, having a substantially flat base or base wall 104 for resting against the skin of a patient and a generally opposed or upper wall 106, and a mouth or mouth portion 108. “Substantially flat” does not exclude a degree of curvature to conform to the general shape of a patient's body, such as upper arm, hip or thigh. The upper wall, as exemplified by FIGS. 1 and 2 is spaced from the base, by reason of a curved or bulbous shape to define a cavity 110 within the housing.

The medical fluid 100F (FIG. 2), such as a vaccine or drug, may be contained within the cavity 108, and in that regard, the housing preferably is made any suitable material, preferably glass or polymer, that is compatible with the medical fluid to be contained therein and for providing an acceptable shelf life. In the illustrated embodiment, the housing or body 102 includes an inner reservoir or vial member, generally at 112, including a resilient expandable container portion or member such as a bladder, balloon or balloon-type member 114 located within the cavity 110, and a penetrable access portion, generally at 116. For containing drugs, vaccines and the m like, the expandable member 114 is preferably made of or has an inner lining of compatible medical grade butyl, silicone or other material suitable for storing the medical fluid. The expandable member is preferably resilient so that it expands upon filling from a relatively small volume to a larger filled volume (as shown in FIG. 2) and exerts an expulsive pressure on the fluid to expel it automatically when the chamber is penetrated or pierced by an access member such as an administration needle or blunt cannula.

The expandable balloon-type container portion or member 114 preferably has a filled volume, as illustrated in FIG. 2, substantially equal to a single dose or unit of the desired vaccine, drug or other medical fluid. Also, the container portion 114 is preferably sufficiently resilient to collapse or deflate to the smaller unfilled volume when the contents are accessed by a needle, and substantially all the medical fluid is forced through the needle under pressure exerted by the stretched wall of the balloon-type container portion or member when it is filled.

The material, wall thickness and other characteristics of the bladder or balloon-type container portion or member 114 may be selected to establish the desired pressure exerted on the fluid, and thereby control the flow rate of the injection, with higher pressures resulting in faster flow rates and lower pressures resulting in slower flow rates. In cooperation with the injection needle size, the flow rate and time required for injection may be varied. For a given expandable member, smaller diameter needle lumen will, of course, result in longer injection times and slower injection flow rates. Larger needle lumen may be employed as desired for rapid injection/inoculation, as deemed appropriate by the medical professional.

For example, for relatively quick injection times of a few seconds, the injection needle lumen diameter is preferably about 0.1 mm or larger. An injection rate of about 0.5 ml/sec is considered a relatively fast injection rate, although needle lumen size could also be selected for a slower injection rate if desired. The needle lumen size could also be smaller or the container portion pressure smaller and provide an injection flow rate less than about 0.5 ml/sec for less painful injection. Injections with a slower flow rate such as about 50 μL/s or less, or about 20 μL/s or less, with injection time of at least several minutes are even less painful and potentially pain free, as recognized in the prior art.

Turning back to the reservoir or vial 100, the mouth 108 is preferably formed by a generally radially extending neck portion 118. The neck portion includes a radially extending flange 120 and a pair of opposed slots 122 extending from the end of the neck partially along the length of the neck, for needle passage therethrough.

The neck is preferably integral with the other portions of the vial or reservoir housing and is made of glass or polymeric material or other suitable rigid material, such as by blow or injection molding in a manner similar to the molding of conventional medication vials. The flange 120 on the neck allows for hermetical sealing in a manufacturing process similar to conventional vials, such as with a foil closure (not shown) sealed around the underside surface of the flange.

For use in this particular embodiment of the vial or reservoir, the inner member 112 also includes a neck portion 124 located within lumen 126 of the radially extending vial neck 118. The neck portion 124 is thicker than the container portion 114 and terminates in a pair of opposed thicker flanges 128 which are received within slots 122, and define an access or septum region through which a needle may be inserted to access the medical fluid within the vial, as described in more detail below. The inner member terminates in an open fill port 130 which may be sealed by a plug or other closure 132, which may also be pierceable if desired.

FIGS. 3-14 illustrate a version of a medical fluid injection device or system particularly suited for use with the vial or reservoir of FIGS. 1 and 2. More specifically, FIG. 3 is a perspective view of a needle assembly or injection device 134. Broadly speaking, the medical fluid injection device or needle assembly has a housing that includes a needle holder or subassembly and a needle shield. More specifically, the illustrated device includes a needle shield 136, a needle holder or needle subassembly 138 and a vial receiving station or recess, such as a vial cover or shell 140, connected, in this embodiment, to the distal end of the needle shield.

More specifically, FIGS. 3 and 4 illustrate the device or needle assembly 134 in a needle-retracted position prior to or subsequent to administration of a medical fluid to a patient. In the retracted position or configuration, the illustrated needle shield 136 is generally elongated and tubular in shape, extending from a proximal end to a distal end and having enlarged formations 142 at the distal end for accommodating the neck portion 118 of vial 100. The shield includes a pair of opposed longitudinally extending sidewalls slots 144, only one of which is visible in FIG. 3, for allowing the separation of the opposing walls of the needle shield, as described in more detail later. The needle shield also includes a pair of opposed proximal latch openings 146 and a pair of opposed distal latch openings 148 that assist in releasably locking or retaining the to needle holder or subassembly 138 in a retracted or injection position, respectively.

Needle holder or subassembly 138 includes an injection needle 150 of suitable length and diameter for the desired injection fluid flow rate, and a needle head or pusher member 152 bonded or otherwise attached to the needle 150, such as at the proximal end of the needle. The injection needle 150 extends from the proximal end, where it is bonded to the pusher 152, to a distal piercing end 154 for injection through the skin of a patient. The illustrated injection needle 150 includes an intermediate access port 156 for accessing the contents of the vial when the needle is moved to the injection position. Although disclosed in the illustrated embodiment as an intermediate access port, depending on the particular configuration of the device, access to the contents of the vial may be provided through the proximal end of the needle (or other access member) or through a different needle configuration wherein the needle lumen accesses the contents of the vial as or after the needle is moved to the injection position. Details of the side access port into the needle interior lumen (which extends at least between the access port 156 and piercing end 154) are illustrated in FIGS. 5a and 5b.

The needle holder or pusher member 152 has a pair of proximally extending, opposed flexible legs 158, which terminate in laterally extending latch projections or ears 160. In cooperation with or extending into the proximal needle latch openings 146, the latch projections 160 serve to hold the injection needle in a retracted position when the needle shield is in the elongated tubular configuration, as shown for example in FIGS. 3 and 4. The needle head or pusher 152 also includes a pair of laterally extending latching projections 162 located on the distal end of the legs 158. The latching projections 162 have inclined or tapered leading surfaces and a proximal undercut or shoulder. The latching projections 162 extend laterally from the pusher member 152 into the sidewall slots 144 of the needle shield, and help to keep the pusher member or needle head and needle in a properly oriented position relative to the vial during movement to the injection position as shown in later figures, such as FIGS. 9-12.

In this embodiment, the needle shield 136 preferably has certain qualities or features to assist in advancement and retraction of the needle to and from an injection position. Preferably the needle shield is collapsible or compressible so as to allow user pressure on the proximal end of the shield to cause the needle 150 to advance distally, and also provides support for the needle alignment during advancement and retraction of the needle. As is apparent from the drawings, in the retracted position, where the shield is essentially of the elongated tubular configuration, the needle is protected against inadvertent touch contamination or needle stick by the user, and in a compressed or collapsed position, the needle is advanced beyond the shield to the injection position (see FIGS. 9-12). In the embodiment illustrated in FIGS. 3 and 4, is the needle shield 136 has at least one annular line of weakness 164 that allows it to fold or collapse into opposing wings when axial pressure is applied at the proximal end of the shield. Additional lines of weakness may also be used, and/or other shapes of the needle shield may be suitable, such as a corrugated or accordion shape, or the needle shield could, for example, take the form of a coil spring with the coils having a pitch such that the needle coils are close enough together to protect the user from inadvertent needle touch contamination or needle stick injuries.

FIGS. 6-8 illustrate the needle assembly of FIGS. 3 and 4 in combination with the medical fluid reservoir or vial 100, pre-filled with a single dose of vaccine, drug or other medical fluid 100F and ready for injection into the patient. The “patient” may be human or other animal, and the subject described herein may have veterinary in addition to human applications. As shown in FIGS. 6-8, the distal end of the needle shield is shaped to fit over the neck 118 of the vial body or housing, and the vial cover or shell 140 nests over the upper wall 106 of the vial 100. The needle 150 may be releasably retained in the retracted position by the resilience of needle shield or sheath, which is inherently biased as a result of the shield construction and materials, to the retracted position of FIGS. 3 and 4. Alternatively or additionally, a biasing structure such as a spring 166 also can be provided within the needle shield or sheath as described below. The user can move the needle to the injection position by depressing or applying axial compressive force against the proximal end of the needle shield. In response to this actuation pressure, the spring, if provided, compresses and the walls of the needle shield fold outwardly along the lines of weakness 164, collapsing to a near-flattened configuration such as may be seen in FIGS. 9 and 11, allowing the needle 150 to move distally, piercing the enlarged seal areas or flanges 128 of the vial inner member 112, which contains the vaccine or other medical fluid. Continued axial force on the proximal end of the needle shield forces the needle holder or pusher member (or needle head) 152 distally or downwardly, with the needle piercing through the skin of the patient, until the latching projections 162 enter into the distal needle latch openings 148 in the wall of the needle shield. The needle projections have inclined or tapered distal surfaces, facilitating a slight spreading action of the needle shield or compression of the projections, allowing the projections to enter the distal latch openings, at which point the edges of the distal latch openings engages against the shoulders of the latching projections to releasably hold or lock the needle and needle holder in the injection position and prevent movement of the needle and shield to the retracted position.

The advanced position may be seen in FIGS. 9-12, which show the needle shield 136 in an outwardly flared, somewhat flattened condition, folded along the line of weakness 164 and separated along the longitudinal slots 144. The internal spring 166, which biases the pusher member or needle holder head 152 to a normally retracted position within the shield, is tightly compressed between an inner surface of the needle shield and the needle head. When the needle is in the fully advanced injection position, the intermediate port 156 of the needle is located in the neck 124 of the inner reservoir or vial member 112 within the mouth 108 of the outer vial body 102, providing direct access to the medical fluid contents 100F of the vial. Pressure exerted by the resilient enlarged expandable container portion 114 forces the medical fluid outwardly through the intermediate opening 156 and through the piercing end 154 of the needle 150 into the patient. After the dosage of medical fluid has been administered, the user can release the retention ears or catches 162 from apertures 148 by direct contact or squeezing projections 160, and the biasing force, such as from the spring 166 or the inherent biasing force of the flared needle shield, moves the needle automatically to the retracted needle-shielded position and the needle assembly and vial may be removed from the patient and discarded. As noted above, the size and length of the injection needle 150 and the pressure of the medical fluid in the vial 100 may be selected for any desired penetration depth or injection fluid flow rate as may be desired for the particular application. For example, administration of vaccines to children may preferably use a shorter needle of smaller diameter and/or lower pressure, so that the injection flow rate is slower and less painful. For mass inoculations where speed is of the essence, larger bore needles and/or higher pressures may be used with higher injection flow rates.

In any event, as noted above, after injection is completed, the user squeezes the opposed proximal latching members 160 of the needle head. This causes the latching projections 162 to move radially inwardly, releasing them from the distal needle latch openings 148. When the latching projections 162 are released from the distal openings, the compressed biasing spring 166 forces the needle holder subassembly (i.e., needle head and associated injection needle) immediately to a retracted position, withdrawing the needle from the patient, and the needle shield unfolds, resuming substantially its original elongated tubular configuration as shown in FIGS. 13 and 14. At this point, the needle piercing end 154 is proximal of the distal end of the shield and is protected from inadvertent contact and the entire needle assembly and empty reservoir or vial 100 may be appropriately discarded, either together or separately.

FIG. 15a is cross-sectional view of another form or configuration of unit dose medical fluid vial 200 in accordance with the subject matter described herein. The illustrated vial, which may be also referred to as reservoir, includes an outer, preferably rigid body 202 generally cylindrical in shape with bottom wall 204 at one end and a neck portion 206 opening at a mouth 208 at the other end of the vial body. For containing medical fluid 200F, the vial 200 includes an inner member generally at 210 which has a resilient expandable container portion 212 and an access portion 214 which normally seals the container portion and may be penetrated for accessing the contents of the container portion.

As can be seen in FIG. 15a, the access portion 214 of the inner member or insert 210 is dimensioned to fit tightly against the inside surface to 216 of the vial neck 206. The access portion also includes a radially extending flange 218 which is seated against the lip of the vial mouth. A crimp or retainer ring 220 holds the inner member in tight sealed contact with the mouth of the vial in a manner well known in pharmaceutical vial manufacture. The crimp ring 220 has a center opening or aperture 222 through which a penetrating member may be inserted for penetrating or piercing the access portion to access the contents of the inner member of the vial.

As shown in FIG. 15a, the resilient container portion 212 has a relatively small volume before filling. A fill port 224 in the access portion allow medical fluid 200F such as vaccines, drugs or the like to be inserted into the container portion. As noted above, the container portion is resilient and expandable, for filling with preferably a unit dose of the selected medical fluid. The resilient nature of the inner member, when stretched to an enlarged position when it is filled, as shown in FIG. 16a, provides an expulsive force on the medical fluid within the container portion. Thus, when the container portion is accessed, the medical fluid is automatically ejected under the force of the resilient container portion. A port or closure plug 226 may be used to seal the fill port after filling with medical fluid. The fill port plug itself may be of a material identical to or similar to that of the access portion of the inner member and may be pierceable or penetrable by an appropriate access member, such as a needle or blunt cannula. As described in the earlier embodiment, any suitable biologically compatible material may be used for the inner or insert member, such as medical grade butyl, silicone, latex or other suitable material. The vial outer body 202 is preferably rigid and may be formulated of rigid plastic, glass or similar materials.

FIGS. 17a-17g illustrate a medical fluid injection device or system (sometimes referred to as an assembly) 228 for injecting fluid from a medical fluid reservoir or vial, such as a vial shown in FIGS. 15 and 16, into a patient. The medical fluid injection device 228, as illustrated, includes a rigid plastic housing generally at 230 that includes a needle holder 232 and a separate needle shield 234. The needle holder, as best seen in FIG. 18, mounts a needle 236 that has opposed piercing ends 238 for penetration through the skin of a patient and 240 for insertion into a vial.

Turning back to FIG. 17, the needle holder and needle shield are generally cylindrical, although other; shapes may be suitable, and the needle shield is slidably received within the needle holder for movement between a needle retracted position as illustrated in FIG. 17, where the needle is located within the shield and protected by the shield from inadvertent contact or touch by a user, and a needle extended position (FIGS. 22a-h), wherein the piercing end of the needle extends beyond the end of the shield for piercing the skin of a patient to inject fluid from a medical fluid vial.

The needle holder 232 is shown in greater detail in FIGS. 18a-18g. As can be seen there, and as explained above, the needle shield is generally cylindrical. It has a side wall portion, generally at 242, with an open upper or proximal end having a radially extending rim or flange 244 and a distal or bottom wall or floor 246.

The needle holder has several independent and distinct features that cooperate to provide significant functionality to the medical fluid injection device described herein. As best seen in FIG. 18a, the needle holder is generally hollow, providing an internal vial-receding recess or bore, generally at 248, that extends between the proximal rim or flange 244 and the floor 246 of the needle holder, for slidably receiving a vial (such as vial 200), mouth end first, into the recess.

The needle holder 232 has three pairs of independently movable flexible fingers of differing length which cooperate with a vial when it is inserted into the vial receiving access. As will be apparent from the drawings, each finger or arm extends distally within an elongated slot in the side wall 242 of the needle holder such that the fingers are attached to the holder only at their proximal end, and the distal ends are free to flex. Specifically, the needle holder includes at least one and preferably a pair of opposed, longitudinally extending fingers or arms 250 (spaced 180° apart) that terminate in radially inwardly extending hook or barb surfaces 252. These are the longest of the three pair of fingers. As will be described in more detail later, the hook surfaces engage against and stop the vial from insertion beyond the loaded position in the vial receiving recess 248. The needle holder also includes at least one and preferably a pair of shorter, longitudinally extending fingers 254 which are inclined or tapered so as to extend a small distance radially into the recess 248. These fingers are sufficiently flexible to allow the end of the vial to move past them as the vial is inserted into the vial loaded stage or position, at which point the fingers 254 snap over a trailing shoulder of the vial neck to substantially prevent withdrawal of the vial from the loaded station. The fingers 254 are the shortest of the three pair of fingers.

The third pair or intermediate length fingers 256 also are inclined or tapered so as to extend slightly radially into the vial receiving recess 248. These fingers are also flexible and flex outwardly to accommodate the end of the vial as it is inserted into the recess. These fingers are of a length to snap over the trailing shoulder of the vial neck or mouth to lock the vial in generally its final position within the housing 230 and prevent withdrawal. Examples of these positions and the function of these fingers will become more apparent in the drawings and description below.

In addition to the fingers, FIG. 18d also depicts one of a pair of cam tracks, grooves or slots 258 that are located 180° apart in the outer surface of the needle holder side wall 242. The cam track cooperates with a protruding pin or follower (not seen in FIG. 18) from the needle shield, as will be described in more detail below. The needle pin and track cooperate to prevent inadvertent separation of the needle holder from the needle shield after assembly and provide an elegant arrangement to control user manipulation of the medical fluid injection device to inject the contents of the vial and return it to a safe shielded position after the injection is complete. As seen in FIG. 18d, and explained more fully below, the cam track has a plurality of positions relative to the pin or follower that correspond to different relative positioning between the needle holder and the needle shield. Specifically, the cam track holder has an initial or first position 260 which corresponds to the needle holder and needle shield in a needle retracted position, a second position 262 that corresponds to the injection position of the needle holder relative to the needle shield and a third position 264 that corresponds to a needle retracted and shielded position.

Turning now to FIGS. 19-27, these figures illustrate the relative position of the needle holder, needle shield, needle and vial during ordinary intended use of the medical fluid injection device assembly described herein.

FIGS. 19a and 19b illustrate the medical fluid injection device 228 as it may be fabricated and shipped in an outer sealed package 266. The outer package includes a generally cylindrical shell or receptacle portion 268 for receiving the needle holder and needle shield and an end seal 270 sealing the receptacle portion 268 and including a pull tab 272 for user removal of the end seal.

FIGS. 19c-19f show the needle holder 232 and needle shield 234 in cross section within the package. As shown there, the needle holder 232 is in a needle retracted position, with the patient piercing end 238 of the needle withdrawn proximally of a distal end wall 274 of the needle shield to protect against touch contamination or needle stick injury from inadvertent user contact. As shown there, the needle is secured within an insert member 276 fixed in the bottom wall 246 of the needle holder. The needle may be bonded by adhesive or employ other means of attachment to the insert member which is, in turn, bonded or otherwise secured to the bottom wall of the needle holder. Alternatively, the needle holder can be bonded or molded directly into the needle holder.

The proximal piercing end 240 of the needle 236 extends into the vial receiving recess or bore 248 of the needle holder 232. Although illustrated with a single needle having opposed piercing ends, it is within the scope of this disclosure that another form of access member may be used for accessing the contents of the container. Such an access member may be a needle separate from the injection needle, a blunt cannula or other penetration member that is in fluid communication with the injection needle to allow the contents of the vial to flow through the needle into the patient. Therefore, although a single needle having opposed piercing ends is particularly advantageous, it is not the only construction that may be used within the scope of this disclosure.

Further shown in FIG. 19c-19f, the medical fluid device 228 includes a biasing member or structure such as coil spring 278 that extends between the lower end or distal end 274 of the needle shield 234 and the underside of the needle holder floor 246. The coil spring is in compression and tends to push the needle holder in a proximal direction, toward a needle retracted position. Although more clearly shown in subsequent drawings, the coil spring cannot separate or push the needle holder completely out of the needle shield because of interference between the cam tracks 258 of the needle shield and cam followers or pins 280 that extend radially inwardly from the inside surface of the needle shield and extend into the cam tracks. More specifically, in the illustrated embodiment, the needle holder has two cam tracks on opposite sides of the needle holder of substantially identical shape, and the needle shield has two radially inwardly extending pins or cam followers 280 that are 180° apart and extend radially inwardly from the inner surface of the needle shield. In the needle retracted position of FIGS. 19a-f, the cam followers or cam pins 280 are located at the first position or station 260 of the cam track (better seen in FIG. 20h) which prevents further separation of the needle holder from the needle shield.

Before turning to other figures, it should be noted that FIGS. 19c-19f are cross-sectional views taken at different angular positions that extend through the fingers 250, 254 and 256, as shown in FIG. 19b. As a result, the various fingers and hooks described earlier are more readily visible in these figures than they were in FIG. 18. For example, section A-A (FIG. 19c) is taken along a line that extends through the shortest fingers 254, section C-C is taken along a line that extends through the longest fingers 250 and section D-D is taken along a line that extends through the intermediate length fingers 256. FIG. 19e, for example, illustrates one of the elongated fingers 250 terminating in a hook surface 252 in clearer sectional view.

In connection with the loading of a pre-filled medical fluid vial into the medical fluid injection device as shown in FIGS. 19c-19f, the upper rim or flange 244 of the needle holder rests on an annular shoulder 282 at the open end of the cylindrical container receptacle 266. The receptacle 266 is preferably of rigid plastic and the receptacle prevents the needle holder 282 from being inadvertently pushed toward the needle shield 234 as a vial is inserted into the vial receiving recess or bore 248 of the needle holder. While this embodiment makes convenient use of the packaging material to prevent inadvertent movement of the needle holder into the needle shield, it should be noted that other types of locking structures may be provided that also prevent such movement. For example, a user removable pin or ring could be placed between opposing surfaces of the needle shield and needle holder to releasably lock or prevent inadvertent movement of the needle shield from the position illustrated in FIGS. 19 during engagement of a vial with the needle holder and, more specifically, during insertion of a vial into the vial receiving recess or bore 248 when a vial is inserted to the loaded position.

FIGS. 20a-20h illustrate the relative position of the needle holder 232 and needle shield 234 within the outer package receptacle 266 when a vial 200, such as that illustrated in FIGS. 15a-16b, is inserted to a loaded position in the vial receiving recess 248. As can be seen in 20c, for example, when the vial 200 is inserted into the loaded position shown there, the shortest flexible fingers 254 flex to allow the mouth of the vial to move along the vial receiving recess and then snap into a radially inward position, with the end of the fingers engaging behind an annular shoulder 286 of the vial, so as to effectively prevent removal of the vial from the loaded position unless the user resorts to extreme measures such as breaking or destroying the needle holder. In the loaded position, as is best seen in FIG. 20f, the longer fingers or arms 250 with end hooks 252 are hooked against the forward surface or edge of the vial to prevent the vial from being moved further into the vial receiving recess from the loaded position as shown in FIGS. 20. In other words, in the loaded position the vial is captured between the shortest fingers 254 which prevent withdrawal from the vial from the loaded position in the receiving recess and the hooks 252 at the end of fingers 250, which temporarily prevent further insertion of the vial 280 into the vial receiving recess. As may be seen in FIGS. 20e and 20h, each cam pin 280 of the needle shield in located in the first station 260 of the cam slot or track 258, and engagement between the cam pin and bottom surface 288 of the cam track at position 260 prevents longitudinal separation of the needle holder from the needle shield.

It should be noted that this positional relationship between the needle holder and needle shield is the same as when they are initially assembled and packaged. Specifically, when the needle holder and needle shield are assembled, the needle shield is positioned so that each cam pin 280 (which extends radially inwardly from the side wall of the needle shield is passed upwardly through an initial axially extending portion 290 of the track 258 and then the needle shield is rotated slightly counterclockwise and separated slightly longitudinally under the bias force of spring 278 to place the cam pin and cam slot in the respective positions shown in FIG. 20h. The track ramp surface 292, together with the biasing force of coil spring 278, tends to hold the cam follower or pin 280 against the track bottom surface 288 and prevent accidental separation of the needle holder and needle shield. Further, it should be noted that although illustrated with the cam track formed in the side wall 242 of the needle holder and the cam pin 280 extending radially inwardly from the side wall of the needle shield, the relative positioning could be reversed and the shape of the cam track otherwise adjusted, as may be desired to further limit the relative motion between the needle holder and needle shield.

Turning now to FIGS. 21a-h, those figures illustrate the medical fluid injection device assembly 228 with a vial or reservoir 284 inserted to the loaded position and the combination of the vial needle holder 232 and needle shield 234 removed from the outer packaging receptacle 266, and ready for injection of the contents of the vial into a patient.

FIG. 22a illustrates the next stage or relative positioning of the needle holder 232 within the needle shield 234. Briefly, after the medical fluid injection device 228 and vial 284 are withdrawn from the outer package, as illustrated in FIG. 22a and after suitable skin swabbing and skin preparation, the distal end of the needle shield is placed directly against the skin of a subject patient. Force F is then applied by the user against the bottom of the vial, as by a hand or thumb, pushing the vial axially inward within the vial receiving recess 248. This advances the vial and the needle holder to the intermediate or pre-injection position illustrated in FIGS. 22a-22h. As illustrated there, in the intermediate position, the vial is still retained within the needle holder between the shortest fingers 254 and the hooks 252 of fingers 250. However, advancement of the needle holder into the needle shield has forced piercing end 238 of the needle 236 to extend through and beyond a central aperture 292 in the bottom wall 274 of the needle shield and into the skin of a patient (not illustrated in FIG. 22). In this position, the biasing coil spring 278 is compressed between the floor 246 of the needle holder and the bottom wall 274 of the needle shield. In this pre-injection or intermediate position, the access end 240 of the needle has not fully penetrated into the container portion 212 of vial 200 and, as best seen in FIGS. 22f, ramp or tapered leading surfaces 294 at the distal end of the elongated fingers 250 are in proximity to ramp or tapered surfaces of raised abutments 296 extending upwardly or proximally from the bottom wall 274 of the needle shield. This engagement will, upon further movement of the needle holder into the needle shield (as discussed later) cause the fingers 250 to flex or spread outwardly, releasing the vial and allowing the vial to move the remaining distance into the vial receiving recess and the needle 236 to fully pierce the access portion 214 of the vial to access the vial contents. The needle shield has opposed slots 297 in registration with fingers 250 to allow the outward flexing described above.

Referring to FIGS. 22e and 22h, in this relative positioning of the needle holder and shield, each cam follower or pin 280 is located along a first linear portion 298 of cam track 258, a relatively short distance, e.g. a few millimeters, from second cam track injection position 262.

Turning now to FIGS. 23a-23h, they show the medical fluid injection device 228 when it has moved from the intermediate or pre-injection position in FIG. 22 to the advanced or injection position by user force F applied to the end 204 of the vial. Specifically, as noted above, the application of force by the user to the end of the vial causes the ramp or tapered surfaces at the end of the elongated fingers 250, to engage the ramp surfaces of the upstanding abutments 296 on the bottom wall of the needle shield. The ramp surfaces of the abutments cause the fingers 250 to spread and the hooks 252 to release the vial for movement to the fully inserted injection position as shown in FIGS. 23a-h. In the injection position, the end of the vial is located in proximity to the floor 246 of the needle holder 232, and the piercing end 240 of the needle (or other access member if used instead of a needle) extends through the access portion 214 of the vial and into the container portion 212 so as to access the vial contents.

Thus, the downward user force F on the bottom 204 of the vial 200 advances the needle holder 232 within the shield 234, releases the vial retaining fingers 280, and simultaneously forces the needle 236 through the access portion. In actual use, the needle holder and vial may move essentially continuously in one movement from the loaded stage through the pre-injection stage and to the injection stage or position. In the injection position, the pressure exerted on the vial contents 200F by the expanded resilient container portion forces the contents of the vial through the needle 236 and into the patient. Also, when the needle holder advances the incremental distance within the needle shield from the position in FIG. 22 to the position FIG. 23, the intermediate retaining fingers 256, which are temporarily flexed outwardly during insertion of the vial, return to their normal inwardly extending position and catch behind the trailing shoulder of the vial mouth, so as to retain the vial in the injection position within the needle holder.

FIGS. 23e and 23h show the relative position of the cam track 258 and cam pins 280 in the injection position. In brief, as the needle holder has moved from the position shown in FIGS. 21a-h to the position shown in FIG. 23a-h, the pin has moved upwardly along the linear portion 298 of the cam track to the position best seen FIG. 23h, abutting against an upper end of the track and received within an upper notch portion 300 of the track wall.

As noted earlier, the position in FIG. 23h is reached as a result of the continued application of force F by the user against the rear or a proximal end 204 of the vial 200. When the user pressure is released, the needle holder and shield separate very slightly to the position shown in FIGS. 24a-24h, which is still part of the second cam track position. Specifically, upon release of the user force, the biasing coil spring 278 raises the needle holder slightly from the bottom wall 274 of the needle shield. The needle 236 remains in the injection position, with the piercing end 238 of the needle inserted into the patient and the piercing end 240 of the needle extending through the access portion 214 and into the container portion 212 of the inner member for accessing the contents of the vial.

The significance of the position illustrated in FIG. 24a-h is the relationship of the cam track 258 and cam pin 280 which function as part of a push-release or click-release feature that allows the user to quickly and easily return the needle holder and needle to a retracted position after the injection is complete. As shown in more detail in FIGS. 24e and 24h, release of the user force from the vial allows the biasing coil springs 278 to push the vial holder upwardly or proximally, and the cam pin 280 to move downwardly from upper notch 300 to a lower notch 302 in the second position 262 of the cam track. It is expected that the vial needle holder and needle shield will remain in the position shown in FIG. 24a-24h until the injection is complete. As noted earlier, the amount of time that the injection requires depends on the size of the needle and the pressure exerted by the resilient expanded container portion 212 of the inner member. Slower injection rates typically result in less pain and, accordingly, for routine injections in children, such as vaccinations, the injection time may take a minute or more. The base of the needle shield may have double-stick tape that has adhesive on one side that adheres to the needle shield and adhesive on the other side that adheres to the skin of the patient. The adhesive can hold the injection device on the skin during extended injections.

FIGS. 25a-25h are identical to FIGS. 24a-24h except that it may be seen that the container portion 212 of the vial inner member 210 has essentially emptied, and returned to a smaller volume configuration, as pressure from the resilient container portion has expressed the medical fluid 200F through the needle 236 and into the patient.

FIGS. 26a-26h illustrate the next operation step of the vial, when user pressure is again applied against the bottom of the vial 200, compressing the coil spring 278 slightly, essentially causing the needle holder 232 to advance slightly within the needle shield 234. In this step, the exertion of the force against the bottom of the vial causes the cam pin 280 to move from its lower notch position shown in FIG. 25h to a pre-released position best seen in FIG. 26h. As shown there, depression of the vial and needle holder causes the cam pin 280 to move upwardly and slightly counterclockwise against a ramp surface 304 at the top of the cam track. The force between the ramp surface and the cam pin causes the needle holder 232 to rotate slightly relative to the needle shield 234, such that the cam pin is moved to the position shown in FIG. 26h at the top of the cam track and immediately above an elongated vertical linear return leg 306 of the cam track.

When the user force F is then released from the end of the vial, the needle holder and needle shield are separated by the biasing force of the coil spring 278 to the position shown in FIGS. 27a-h. In other words, when the force against the bottom 204 of the vial 200 is released, the cam pin 280 moves directly down the vertical return portion 306 of the track as the coil spring pushes the needle holder upwardly or proximally. The spring force causes the cam pin to be forced against the bottom of the return leg which has a downward angular ramp 308 that relatively rotates the needle holder and shield very slightly and directs the cam pin into a terminal portion 264 of the cam track, as best seen in FIG. 27h. Referring to FIGS. 27a-27g, it may be seen that in this position, the needle holder 232 has been pushed upwardly or proximally by the biasing coil spring 278, withdrawing the needle from the patient and locating it fully within the needle shield 234 where the skin piercing end 238 of the needle 236 is protected from inadvertent user contact. The location of the cam pin within the terminal portion 266 of the cam track, which is held there by the biasing force of spring 278, prevents the needle holder from being inadvertently reinserted into the needle shield also. Also, the intermediate length fingers 256 in the needle holder continue to prevent the vial from being withdrawn from the needle holder. Accordingly, the assembly as shown in FIGS. 27a and 27c-g cannot be separated, except upon user abuse or destruction and is ready for safe disposal in a sharps container or other arrangement. Turning to another aspect of the present subject matter illustrated in FIGS. 28-31, FIGS. 28a and b show a needle holder 400 that has a generally cylindrical body 401 with an open proximal end 402 that defines a vial receiving recess 404 and a distal or bottom wall 406. A radial flange 407 extends outwardly around the body. The bottom wall 406 mounts a double ended needle 408 with distal piercing end 410 for injecting into a patient and a proximal piercing end 412 extending into the vial receiving recess. Retainer arms 414 extend upwardly from the bottom wall for holding a medical fluid vial in an accessed position, as will be discussed later.

FIGS. 29a and b show the needle holder with a prefilled medical fluid vial 416 inserted to an insertion or loaded position in the vial receiving recess 404. The vial 416 is comparable in material respects to the vial 200 illustrated in FIGS. 15 and 16, the description of which will not be repeated. In this position the needle inserted into the patient and is ready to inject the medical fluids.

FIGS. 30a and b show the vial 416 advances to the access position within the vial receiving recess 404. At that position, the proximal piercing end 412 of needle 408 (or other access member) has penetrated the vial septum or access area and the medical fluid can flow through the needle under pressure from within the vial. At this stage or position, the needle protector or shield 418 (FIG. 28b) has been removed and the needle has been inserted into patient's tissue and the drug is being delivered. Also insertion of the vial has caused the retainer arms 414 to spread and the hooked ends of the arms to catch behind the trailing shoulder 420 of the vial mouth to prevent withdrawal of the vial.

FIGS. 31a and b are identical to FIGS. 30a and b, but show the vial inner member 422 deflated after injection of the contents. At this point the vial and needle holder assembly may be discarded.

Although the subject matter here is illustrated in different embodiments, various changes or variations may be apparent from reading this description. For example, although the figures show certain needle assemblies used with certain vials, it should be understood that the needle assemblies may be used with the other vials, and vice versa, as appropriate, with only such design modification as may be needed. Accordingly, it is respectfully submitted that the scope of this disclosure is measured in accordance with the attached claims and not the particular embodiments or versions of the subject matter described herein.

1.-34. (canceled)

35. A single use medical fluid injection system for injecting the contents of a medical fluid vial into a patient, the injection device comprising:
a housing configured for cooperative engagement with a medical fluid vial;
a vial access member within the housing for accessing the contents of the medical fluid vial; and
an injection needle having a piercing end for piercing the skin of a patient, the injection needle being in fluid communication with the access member and movably carried by the housing for movement between a retracted position in which the housing shields the piercing end of the needle against inadvertent touch and an injection position in which the piercing end of the needle extends beyond the housing for piercing the skin of a patient;
the housing defining a plurality of sequential operative stages, including:
a load stage in which a vial is brought into cooperative engagement with the housing, and in which first stage the housing engages the vial and substantially prevents it from being disengaged from the housing, the access member does not access the contents of the vial and the piercing end of the injection needle is located in the retracted position;
an injection stage in which other stage the access member accesses the contents of the vial, allowing the contents to flow from the vial through the access member and the injection needle, and the injection needle is in an injection position for administering medical fluid from the vial into a patient; and
a retracted stage in which the injection needle is retracted and is shielded by the housing from inadvertent touch.

36. The system of claim 35 further comprising:
a medical fluid vial including:
an outer vial body having a mouth; and
an inner vial member comprising a resilient expandable container portion located within the outer vial body for containing medical fluid and exerting pressure thereon and an access portion located in the vial mouth, the access portion normally sealing the container portion and being penetrable for accessing medical fluid in the vial.

37. The medical fluid injection system of claim 35 wherein the vial access member comprises a needle or a blunt cannula.

38. The medical fluid injection system of claim 35 further comprising a releasable prevention member to prevent the injection needle from inadvertently moving from the retracted position to the injection position during engagement of the vial with the housing.

39. The medical fluid injection system of claim 35 further comprising a releasable securing member that prevents movement of the injection needle from the injection position to the retracted position.

40. The medical fluid injection system of claim 35 further comprising a releasable stop that prevents access of vial contents by the access member in the load stage and a stop release which cooperates with and releases the stop upon advancement to the injection stage and allows access of the vial contents by the access member.

41. The medical fluid injection system of claim 35 in which the injection needle is releasably secured in the injection position by a securing member and the securing member is user manipulable to release the injection needle, the injection needle being biased for automatic movement toward the retracted position upon user release.

42. The medical fluid injection system of claim 35 in which the housing comprises a needle shield and a needle holder that are relatively movable.

43. The medical fluid injection system of claim 35 including an intermediate stage between the load stage and the injection stage in which the injection needle is in the injection position and the access member does not access the contents of the vial.

44. The medical fluid injection system of claim 35 comprising a double-ended cannula having one end defining the access member and configured to access the vial contents and the opposite end defining the injection needle and configured to pierce skin.

45. The medical fluid system of claim 35 in which the housing comprises a needle holder and a needle shield that are relatively axially movable between a first relative position in the load stage and a second relative position in the injection stage.

46. The medical fluid system of claim 43 in which the housing comprises a needle holder and a needle shield that are relatively axially movable between a first relative position in the load stage, a second relative position in the intermediate and injection stages, and the needle holder is configured to receive a medical fluid vial and allow movement of the vial relative to the needle holder between a vial contents unaccessed position in the intermediate stage and a vial contents accessed position in the injection stage.

47. The medical fluid injection system of claim 36 wherein the outer vial body has an open mouth at one end thereof and the access portion of the inner vial member includes a filling orifice for introducing medical fluid into the container portion and a pierceable plug for sealing the orifice after filling.

48. The medical fluid injection system of claim 36 in which the resilient body portion has an unfilled volume and is expanded upon filing with medical fluid so as to exert pressure on the medical fluid, which pressure can expel the medical fluid from the container portion without other manipulation upon penetration of the access portion.

49. The medical fluid injection system of claim 48 in which the injection needle is configured to inject medical fluid a rate that does not create substantial user pain.

50. A single use medical fluid vial comprising:
an outer body having an open mouth at one end thereof; and
an inner vial member comprising a resilient expandable container portion located within the vial body and an access portion disposed within the vial mouth, the access portion normally sealing the container portion and providing an access area for withdrawing any contents of the container portion.

51. The medical fluid vial of claim 50 wherein the access portion includes a pre-formed, normally closed aperture for receiving a blunt cannula to penetrate the access portion.

52. The medical fluid vial of claim 50 wherein the access portion includes a filling orifice for introducing medical fluid into the container portion and a pierceable plug for sealing the orifice after filling.

53. The medical fluid vial of claim 50 in which the resilient body portion has an unfilled volume and is expanded upon filing with medical fluid so as to exert pressure on the medical fluid, allowing the medical fluid to be expelled from the container portion without other manipulation upon penetration of the access portion.

54. The medical fluid vial of claim 50 comprising a unit dose of medical fluid.

55. The medical fluid vial of claim 50 in which the inner vial member is of one-piece construction.
